(19) United States
(12) Patent Application Publication
(10) Pub. No.: US 2025/0102492 A1
(43) Pub. Date: Mar. 27, 2025

(54) GAS MEASURING DEVICE AND GAS MEASURING PROCESS

(71) Applicant: Dräger Safety AG & Co. KGaA, Lübeck (DE)

(72) Inventors: Jens REKOW, Lübeck (DE); Malte BAESLER, Lübeck (DE); Peter KAHNS, Lübeck (DE); Tobias REIER, Lübeck (DE); Luis BRUNSWICK FRANCO, Lübeck (DE)

(21) Appl. No.: 18/894,139

(22) Filed: Sep. 24, 2024

(30) Foreign Application Priority Data
Sep. 26, 2023 (DE) 10 2023 125 988.1

(51) Int. Cl. G01N 33/497; A61B 5/08

(52) U.S. Cl. CPC G01N 33/4972; A61B 5/082

(57) ABSTRACT

A gas measuring device (100) and a gas measuring process measure a concentration of a component in a gas mixture (A), in particular the concentration of breath alcohol in a breath sample. The gas mixture (A) flows into a tubular input unit (1) which is detachably connected to a base body (6, 16). A portion of the gas mixture (A) is branched off and the gas sample (Gp) thus produced flows into a measuring chamber (2). A gas sensor (50) measures the concentration of the component in the gas sample (Gp). The gas sample (Gp) is branched off from the input unit (1) at an entry point (P.e) and re-enters the input unit (1) or a channel leading into the environment at a downstream exit point (P.a1). The measuring chamber (2) is connected to these two points (P.e, P.a1) by two different fluid guide units (3, 4).

### CROSS REFERENCE TO RELATED APPLICATIONS

This application claims the benefit of priority under 35 U.S.C. § 119 of German Application 10 2023 125 988.1, filed Sep. 26, 2023, the entire contents of which are incorporated herein by reference.

### TECHNICAL FIELD

The invention relates to a gas measuring device and a gas measuring process which are capable of measuring the concentration of a component in a gas mixture.

### BACKGROUND

The task of measuring the concentration of a component in a gas mixture occurs, for example, when a test subject is to be examined to determine whether they have consumed alcohol. If they have consumed alcohol, the air they exhale contains breath alcohol. In this application, the gas mixture is a breath sample given by the subject and the component which concentration is to be measured is breath alcohol. The component can also be another substance that can be detected in a breath sample.

### SUMMARY

It is an object of the invention to provide a gas measuring device and a gas measuring process which comprise a measuring chamber for receiving a gas sample and which are capable of guiding a gas sample towards and away from the measuring chamber with greater reliability than known devices and processes.

The problem is solved by a gas measuring device with features according to the invention and by a gas measuring process with features according to the invention. Advantageous embodiments of the gas measuring device according to the invention are, where appropriate, also advantageous embodiments of the gas measuring process according to the invention and vice versa.

The terms “fluid guide unit” and “fluid conveying unit” are used below. A fluid guide unit is a component that is able to guide a fluid along a trajectory wherein the trajectory is specified by the geometry, configuration and/or arrangement of the fluid guide unit. Ideally the fluid guide unit prevents the guided fluid from leaving this trajectory. A hose and a tube are two examples of a fluid guide unit. A fluid conveying unit is capable of conveying a fluid, i.e. moving it, in particular through a fluid guide unit. A pump, a blower and a piston-cylinder unit are examples of a fluid conveying unit.

The gas measuring device according to the invention and the gas measuring process according to the invention are capable of measuring the concentration of at least one component in a gas mixture. The gas mixture is, for example, breath given by a subject and the component which concentration is to be measured is breath alcohol or another substance which may be contained in a breath sample and which is to be detected. The gas mixture can also come from a source other than a subject's mouth.

The gas measuring device according to the invention comprises a tubular input unit, wherein the input unit has an inlet opening and an outlet opening. Preferably, the input unit extends along a longitudinal axis. It is possible that the input unit tapers seen in a direction from the inlet opening towards the outlet opening.

Furthermore, the gas measuring device comprises a base body. The input unit is connected to the base body or can be connected to the base body, preferably detachably. A detachable connection makes it possible to replace the input unit after inputting a gas sample and to continue using the base body.

At least one measuring chamber and a sensor arrangement comprising at least one gas sensor of the gas measuring device are arranged inside the base body. Optionally the sensor arrangement comprises several gas sensors, in one embodiment several measuring chambers and several gas sensors are provided. An embodiment with one measuring chamber and one gas sensor is described below. This description also applies accordingly to an embodiment with several gas sensors and/or several measuring chambers.

The measuring chamber can accommodate a gas sample. The base body has an entry point (entry region) and an exit point (exit region), whereby these two points are spaced apart from each other. A feed fluid guide unit connects the entry point with the measuring chamber. A discharge fluid guide unit connects the measuring chamber with the exit point. These two fluid guide units are arranged at a distance from each other and without crossing in the base body. Preferably, these two fluid guide units or at least one respective segment of each of these two fluid guide units extend along two longitudinal axes, whereby the two longitudinal axes each form an angle of at least 60° with the longitudinal axis of the input unit and are particularly preferably both perpendicular to the longitudinal axis of the input unit.

The gas measuring process according to the invention is carried out using such a gas measuring device. The gas measuring device is adapted to perform the following steps, and the gas measuring process comprises the following corresponding steps:
The input unit is connected to the base body, preferably releasably connected.
A gas mixture, e.g. a breath sample, flows through the inlet opening into the input unit.
At the entry point, a gas sample is branched off (diverted, taken) from the gas mixture flowing through the input unit. The remainder (rest) of the gas mixture, i.e. the gas mixture without the branched-off gas sample, flows past the entry point and then past the exit point and then leaves the input unit through the outlet opening. Preferably, a maximum of 10% by volume of the gas mixture is branched off as a gas sample.
The branched-off gas sample flows through the feed fluid guide unit into the measuring chamber.
The branched-off gas sample flows out of the measuring chamber and through the discharge fluid guide unit to the exit point and there out of the base body.

According to a first alternative, the gas measuring device is configured as follows, and the gas measuring process comprises the following steps:
The branched-off gas sample flows at the exit point back into the input unit.
The entire gas mixture, including the branched-off gas sample, leaves the input unit through the outlet opening.

According to a second alternative, the gas measuring device is configured as follows, and the gas measuring process comprises the following steps:
At the exit point, the branched-off gas sample flows out of the base body and, after exiting the base body, flows past (along) the input unit and then into an area surrounding the gas measuring device.
While the branched-off gas sample flows past the input unit, it flows in a direction away from the inlet opening and preferably in a direction parallel to a longitudinal axis of the input unit and thus parallel to the rest of the gas mixture, i.e. to the part of the gas mixture that has not been branched off.

In both alternatives, both the branched-off gas sample and the rest of the gas mixture preferably flow back into an environment of the gas measuring device.

The gas sensor or each gas sensor is configured to measure the concentration of the component in the gas sample while the gas sample is in the measuring chamber. More precisely, the gas sensor or each gas sensor is capable of measuring an indicator that correlates with the concentration of the component. In the case of at least two gas sensors, these two gas sensors preferably measure two different indicators, both of which correlate with the concentration of the component.

The terms “upstream” and “downstream” as well as “left” and “right” are used below. These terms refer to the flow direction in which a gas mixture flows through the input unit and have the usual meanings in relation to this direction. The terms “up” and “down” refer to an orientation in which a user holds the base body in one hand and the input unit is above the base body. In this application, downstream refers to a direction away from the user.

According to the invention, the gas mixture flows through the inlet opening into the tubular input unit. At least that part of the gas mixture which is not branched off at the entry point leaves the input unit through the outlet opening. The branched-off part, i.e. the gas sample, leaves the base body through the exit point. According to the first alternative, the branched-off part flows back into the input unit. According to the second alternative, the branched-off part flows past the input unit and in a direction away from the inlet opening of the input unit.

In many cases, the gas measuring device is held by a human being, for example because this human being or another human being inputs the gas mixture into the input unit. The feature just described, in conjunction with the tubular shape of the input unit, reduces the risk of the gas mixture being blown into a human being's face or flowing into the human being's face or otherwise unintentionally reaching a human being after the gas mixture has flowed through the input unit. The base body can be aligned so that the outlet opening of the input unit does not point towards a human being. The undesirable event of the gas mixture flowing into a human being's face can be prevented without necessarily having a retaining element in the input unit.

It is undesirable that the escaping gas mixture reaches a human being, in particular because the gas mixture may contain pathogens, which is particularly possible if the gas mixture is exhaled breath. Therefore, according to the invention the gas mixture is directed away from the gas measuring device in a defined manner.

As a rule, the input unit comprises an additional opening in addition to the inlet opening and the outlet opening, this additional opening acting as the entry point where the gas sample is branched off (diverted/tapped). The second alternative avoids the need for an additional opening in the wall of the input unit in addition to the entry point, with this additional opening acting as the exit point. Two openings in a wall of the input unit will in some cases lead to more turbulence in the input unit than just one opening.

As a rule, the branched-off gas sample changes its flow direction after the gas sample is branched off from the input unit and while the gas sample flows through the feed fluid guide unit. This feature reduces the risk of substances entering the measuring chamber that are harmful to the measuring chamber or the gas sensor. In particular, it reduces the risk of water in the gas sample condensing on a surface of the measuring chamber or a surface of a sensor or of larger particles from the environment or the input unit entering the measuring chamber. This risk would be greater if the gas mixture were to flow from the inlet opening into the measuring chamber without changing its flow direction.

According to the invention, the branched-off gas sample flows through the feed fluid guide unit into the measuring chamber and through the discharge fluid guide unit out of the measuring chamber. The invention eliminates the need for a gas sample to flow first into the measuring chamber and then out of the measuring chamber through the same fluid guide unit. The embodiment with a single fluid guide unit or with two intersecting fluid guide units can lead to a backflow or backlog in the single fluid guide unit or at the intersection point. If the gas sample is sucked in by a piston, this piston must in many cases cover a relatively long stroke in the case of a single fluid guide unit and therefore pass along a quite long way. The invention makes it possible to branch off the gas sample using a relatively small fluid conveying unit and then expel the gas sample from the measuring chamber. A continuous measurement, which is theoretically unlimited in time, is made possible. When using a piston in conjunction with a single fluid conveying unit, the measurement would inevitably have to be interrupted frequently.

According to the invention, the gas sample flows from the measuring chamber through the discharge fluid guide unit to the exit point and exits the discharge fluid guide unit at the exit point. According to the first alternative, the gas sample flows back into the input unit at the exit point, so that the entire gas mixture exits through the outlet opening of the input unit.

In the second alternative, the branched-off gas sample and the remainder of the gas mixture generally escape into the environment at two points spaced apart from one another. According to one embodiment of the second alternative, the gas sample mixes in a mixing region, which is arranged downstream of the outlet opening of the input unit, with that part (the remainder) of the gas mixture which has flowed through the input unit without being branched off.

In one embodiment of the embodiment with the mixing region, a channel is formed between the input unit and the base body when the input unit is connected to the base body. This channel connects the exit point with the mixing region. The mixing region belongs to the surroundings of the gas measuring device or is in a fluid connection with the surroundings. Preferably, the channel extends parallel to a longitudinal axis of the tubular input unit. The gas sample flows from the measuring chamber through the discharge fluid guide unit to the exit point and then from the exit point through this channel and thus past the input unit to the mixing region.

In some cases, the second alternative and in particular the embodiment just described reduce the risk of undesirable turbulence occurring in the input unit and/or of a backflow, backlog, or back pressure occurring in the discharge fluid guide unit. The channel guides the gas mixture away from the inlet opening of the input unit with particularly high reliability. This further reduces the risk of a part of the gas mixture flowing into the face of a human wherein the input unit is located in front of this face.

It is possible that the outlet opening of the input unit is flush with the base body. It is also possible that the input unit protrudes upstream and/or downstream of the base body. In one embodiment, on the other hand, the base body protrudes over the input unit downstream of the input unit. Downstream of the outlet opening, a region is formed which is provided and limited on one side by the input unit, in particular by the outlet opening, and on another side by the part of the base body projecting downstream. In this region, the entire gas mixture passes through the outlet opening in the first alternative. In the second alternative, the branched-off gas sample passes through the exit point and past the input unit into this region and the rest of the gas mixture passes through the outlet opening into the region. This embodiment can be combined with the implementation of the second alternative just described according to which a channel between the base body and the input unit leads to the mixing region.

In one embodiment, a separating element divides the input unit into a chamber on the inlet side and a chamber on the outlet side. The inlet opening adjoins the inlet-side chamber, the outlet opening adjoins the outlet-side chamber. At least one passage opening is recessed into this separating element. Both the entry point and the exit point are adjacent to the chamber on the outlet side. The separating element separates the chamber on the outlet side from the inlet opening. The entire gas mixture flows through the inlet opening and through the inlet-side chamber to the separating element. At least part of the gas mixture flows through the passage opening in the separating element into the outlet-side chamber.

The gas sample is therefore branched off out of the inlet unit downstream of the separating element and in the outlet-side chamber. Compared to a configuration without a separating element in the input unit, this configuration reduces the risk of turbulence or a very high overpressure or vacuum relative to the ambient pressure occurring in the outlet-side chamber. This makes it easier to divert (branch off) a gas sample with a relatively uniform volume flow.

The separating element can be configured as a purely passive mechanical component. In one embodiment, the inlet-side chamber extends along the entire length of the input unit, i.e. from the inlet opening to the outlet opening. When viewing in a flow direction of the gas mixture through the input unit, a segment of the inlet-side chamber is located adjacent to or above the outlet-side chamber. This implementation further reduces the risk of turbulence or a large vacuum or overpressure occurring in the outlet-side chamber.

Preferably, the input unit can be detachably connected to the base body and separated from the base body again. In one embodiment, the gas measuring device comprises a pressure sensor. This pressure sensor is able to measure the pressure in a fluid guide unit-more precisely: directly measure the pressure or measure an indicator that correlates with the pressure. In one embodiment, this fluid guide unit is the feed fluid guide unit; in another embodiment, it is the discharge fluid guide unit. Depending on the measured pressure, the gas measuring device—or more precisely: a signal-processing control unit or other device of the gas measuring device—can automatically decide whether the input unit is connected to the base body or not. When the input unit is attached, the pressure is usually significantly higher, i.e. above a specified pressure threshold, than when the input unit is not attached. This significant difference occurs at least while a fluid is flowing through the fluid guide unit, in particular while a gas mixture is being fed into the input unit or while the measuring chamber is being flushed out.

This configuration reduces the risk of one of the following undesirable events occurring without being detected:
According to the embodiment just described the pressure sensor is configured to measure the pressure in a fluid guiding unit. Ambient air flows through this fluid guide unit into the measuring chamber or out of the measuring chamber. As a result, the measuring chamber is flushed out after a measurement and is available for a new measurement after flushing. This flushing is a desired process. During flushing, no input unit should be connected to the base body. Without an input unit on the base body the measuring chamber is actually flushed out completely.
Although the gas measuring device is now to be used to test a gas mixture for the component, no input unit is connected to the base body. This is obviously a mistake.

Preferably, the gas measuring device generates a message when it has detected one of these two undesirable events. This message is output in a form that can be perceived by a human being, preferably by an own output unit of the gas measuring device.

According to the invention, a part of the gas mixture flowing through the input unit is branched off and fed as a gas sample through the feed fluid guide unit into the measuring chamber. In one embodiment, the kinetic and/or potential energy of the gas mixture flowing into the input unit is sufficient to divert (branch off) the gas mixture. This applies, for example, if the gas mixture is a breath sample that a test subject gives off, provided that the test subject exhales sufficiently strongly. It is possible that the gas measuring device does not have its own fluid conveying unit.

In one embodiment, however, the gas measuring device comprises a fluid conveying unit, for example a pump or a piston-cylinder unit or a blower. The fluid conveying unit can be switched on and off. The fluid conveying unit is at least capable of conveying the gas sample out of the measuring chamber and through the discharge fluid guide unit. As a rule, gas then flows through the feed fluid guide unit into the measuring chamber, in particular because a vacuum is generated in the measuring chamber. This gas flushes out the measuring chamber and a new gas sample is sucked into the measuring chamber. In one embodiment, the fluid conveying unit also contributes to or even exclusively causes the gas sample to be branched off from the gas mixture flowing through the input unit. In this implementation, the fluid conveying unit preferably sucks the gas sample through the feed fluid guide unit into the measuring chamber.

The embodiment with the fluid conveying unit can be combined with the embodiment in which a pressure sensor measures an indicator of the pressure in a fluid conveying unit. In this combination, the pressure sensor measures an indicator of the pressure in the discharge fluid guide unit. Based on the measured pressure, i.e. based on a signal from the pressure sensor, the gas measuring device can automatically decide whether the fluid conveying unit is switched on or off. As a rule, the fluid conveying unit should be switched on at least temporarily when a gas mixture is fed into the input unit.

The embodiment just described can be combined with the embodiment in which the input unit can be detachably connected to the base body and separated from the base body again. According to this combination, the gas measuring device is able to automatically distinguish between the following three situations:
The fluid conveying unit is switched off (low pressure).
The fluid conveying unit is switched on and the input unit is not connected to the main body (medium pressure).
The fluid conveying unit is switched on and the input unit is connected to the main body (high pressure).

In some cases, the gas measuring device can also detect the following undesirable situation: The fluid conveying unit is switched off, but a pressure between the low pressure and the medium pressure is present in the discharge fluid guide unit. This can be an indication that a subject is entering a breath sample into the input unit even though the fluid conveying unit is switched off. This is often undesirable.

In another embodiment, the gas measuring device uses the signal from the pressure sensor to measure an indicator of a volume flow, with the fluid conveying unit achieving this volume flow. The volume flow is the volume per unit time that flows through a fluid conveying unit. Knowledge of the volume flow can be used, for example, to control the fluid conveying unit with the control objective that the actual and generally time-varying volume flow follows a predetermined time course, in particular the volume flow should be constant over time. The two embodiments,
that the signal from the pressure sensor is used to distinguish the three situations with the detachable input unit and the fluid conveying unit that can be switched on and off, and
that the signal is used to measure the indictor of the volume flow, can be combined with each other. In other words, the signal from the pressure sensor can be used for the two different purposes just described.

In one embodiment, the fluid conveying unit just described comprises the following components:
a chamber whose volume can be changed,
an actuator or a motor,
an intake non-return valve (check valve) and
an output non-return valve.

The actuator is able to change the volume of the chamber. For example, the chamber is limited by a diaphragm or other movable wall, and the actuator is mechanically connected to this movable wall. The chamber may comprise a bellows.

An intake fluid guide unit connects the chamber of the fluid conveying unit with the measuring chamber. An output fluid guide unit connects the chamber to the discharge fluid guide unit. As a result, the chamber of the fluid conveying unit is in an intake fluid connection with the measuring chamber and in an output fluid connection with the discharge fluid guide unit. The intake non-return valve allows fluid to flow through the intake fluid connection into the chamber of the fluid conveying unit and prevents a flow in the opposite direction. The output non-return valve allows fluid to flow through the output fluid connection into the discharge fluid guide unit and prevents flow in the opposite direction.

In this embodiment, the fluid conveying unit can be used both to divert a gas sample from the gas mixture flowing through the input unit and to convey it into the measuring chamber, as well as to expel the gas sample from the measuring chamber and thereby flush out the measuring chamber. In many cases, this arrangement makes it possible to realize a fluid conveying unit that requires relatively little installation space and/or consumes relatively little electrical energy. In many cases, this arrangement also prevents gas from flowing in the opposite direction through the measuring chamber in an undesirable manner.

According to the invention, the gas sensor or each gas sensor of the gas measuring device is able to measure the concentration and/or the proportion (share) of the component in the gas sample in the measuring chamber. In one embodiment, the or a gas sensor is configured as a photoelectric sensor. The gas sensor comprises a radiation source and a photodetector. The radiation source is capable of emitting electromagnetic radiation. The emitted electromagnetic radiation penetrates the measuring chamber and strikes (impinges onto) the photodetector. The photodetector is able to generate a signal depending on the intensity of the incident (impinging) radiation and is therefore a photoelectric detector. The generated signal contains information about the concentration of the component of the gas sample in the measuring chamber.

A gas sensor configured in this way is able to provide a signal about the current concentration of the component continuously, i.e. with a high sampling frequency. In addition, such a sensor does not consume any chemicals and is in many cases less sensitive than other gas sensors to substances that penetrate the measuring chamber and less sensitive to high temperatures that can take a significant influence on a detection variable or lead to evaporation of a chemical. Therefore, in many cases it is not necessary to seal the measuring chamber fluid-tight against the environment when the gas measuring device is not in use.

In a further embodiment of this design, the gas measuring device comprises an electrochemical sensor in addition to the photoelectric sensor. In a first alternative of the embodiment, the gas measuring device is configured so that the branched-off gas sample reaches both sensors one after the other. The two sensors are therefore connected in series. In another alternative, the gas measuring device is configured so that an additional gas sample is branched off the input unit and the additional gas sample reaches the electrochemical sensor. Or the branched-off gas sample is divided between the two sensors. The two sensors are therefore connected in parallel.

The additional gas sensor has an electrical detection variable. During operation, a chemical reaction takes place in the additional gas sensor. The chemical reaction that takes place influences the electrical detection variable. The chemical reaction and therefore the electrical detection variable depend on the concentration of the component in the gas sample or in the additional gas sample. The electrochemical sensor is able to measure the electrical detection variable. The electrochemical sensor is able to generate a signal, the generation of the signal depending on the measured electrical detection variable. The generated signal comprises information about the concentration of the component in the gas sample or in the additional gas sample. In one embodiment, the component to be detected in the gas mixture is an oxidizable (combustible) component, and the chemical reaction causes an electric current to flow. The chemical reaction corresponds to the chemical reaction that takes place in a fuel cell.

The configuration with two gas sensors that use different measuring principles provides redundancy. If one gas sensor fails, the gas measuring device with the other gas sensor can in many cases still be used. In addition, when using two gas sensors a plausibility check can be carried out. If the two sensors provide significantly different measurement results for the same component of the gas sample, this is an indication that at least one gas sensor is defective or may be defective or that a fluid guide unit is blocked. In addition, a gas measuring device with two sensors that use different measuring principles is often able to reliably measure at least two different components in the same gas mixture.

In a preferred embodiment, the gas measuring device according to the invention is capable of detecting the presence and/or concentration of alcohol in the input gas mixture. In this application, the gas mixture is a breath sample from a test subject. In this embodiment, the gas sensor or each gas sensor is capable of measuring an indicator of the concentration of breath alcohol in the breath sample. The gas measuring device is therefore an alcohol measuring device (also known as a breathalyzer). In one embodiment, the alcohol measuring device comprises the two sensors described above, namely the photoelectric sensor and the electro-chemical sensor.

Preferably, the gas measuring device is configured as a portable device. Particularly preferably, the input unit can be held in front of the mouth of a test subject in such a way that the inlet opening points towards the test subject. Preferably, the gas measuring device comprises its own power supply unit in order to be independent of a stationary power supply network. Preferably, the gas measuring device is able to output the result of the measurement on its own output unit.

The invention is described below with reference to embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of the alcohol measuring device from the right;

FIG. 2 is a side view of the alcohol measuring device of FIG. 1, with the base body and the fluid conveying unit omitted;

FIG. 3 is a side view of the alcohol measuring device from the left;

FIG. 4 is a perspective view of the alcohol measuring device viewed from the left and diagonally from below;

FIG. 5 is a partially cutaway perspective sectional view;

FIG. 6 is a perspective view of the alcohol measuring device viewed diagonally from behind from the left;

FIG. 7 is a top view of the mouthpiece and the cover;

FIG. 8 is a top view of the cover with the mouthpiece removed;

FIG. 9 is a perspective sectional view through the gas measuring device, looking diagonally from behind; and

FIG. 10 is a schematic view of a fluid conveying unit in the form of a diaphragm pump.

### DESCRIPTION OF PREFERRED EMBODIMENTS

Referring to the drawings, in the embodiment shown, the invention is used to test whether a test subject has consumed alcohol. As is known, the air that a human being exhales contains alcohol if the subject has previously consumed alcohol and therefore alcohol is present in his or her blood. The invention is used, for example, to test a vehicle driver or plant operator for breath alcohol.

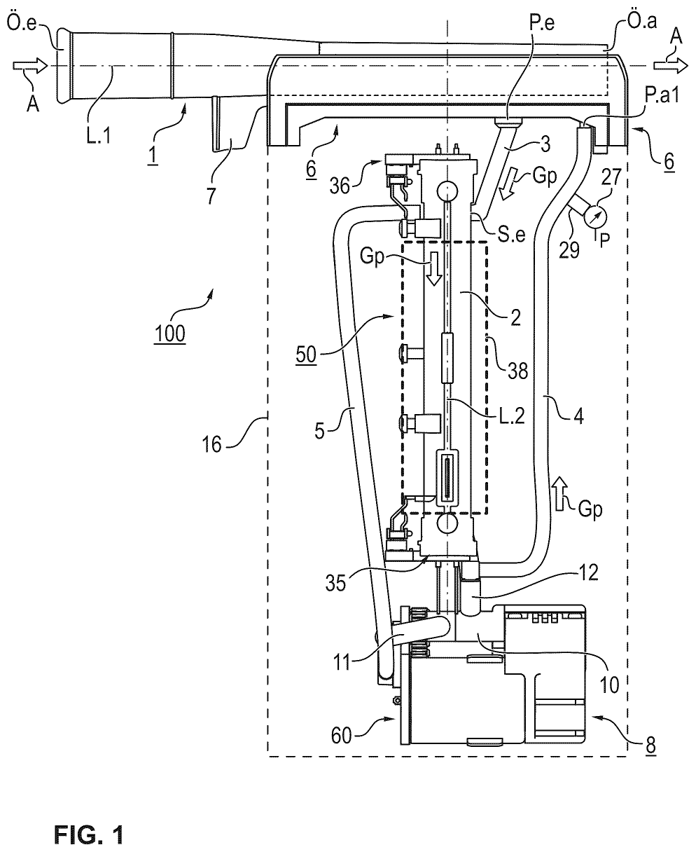

FIG. 1 to FIG. 9 show a portable alcohol measuring device (breathalyzer) 100, in which the invention is used, or a detailed section from different viewing directions. A human being, for example a police officer or the test subject himself/herself, holds the alcohol measuring device 100 in one hand. The test subject (not shown) inputs a breath sample A into a tubular mouthpiece 1. The mouthpiece 1 acts as the input unit, extending along a longitudinal axis L.1 and tapering away from the test subject. The delivered breath sample A enters the mouthpiece 1 through an inlet opening Ö.e. At least part of the breath sample A exits the mouthpiece 1 again through an outlet opening Ö.a, wherein the outlet opening Ö.a is smaller than the inlet opening Ö.e. The terms “left”, “right”, “upstream” and “downstream” used below refer to the flow direction of the breath sample A through the mouthpiece 1.

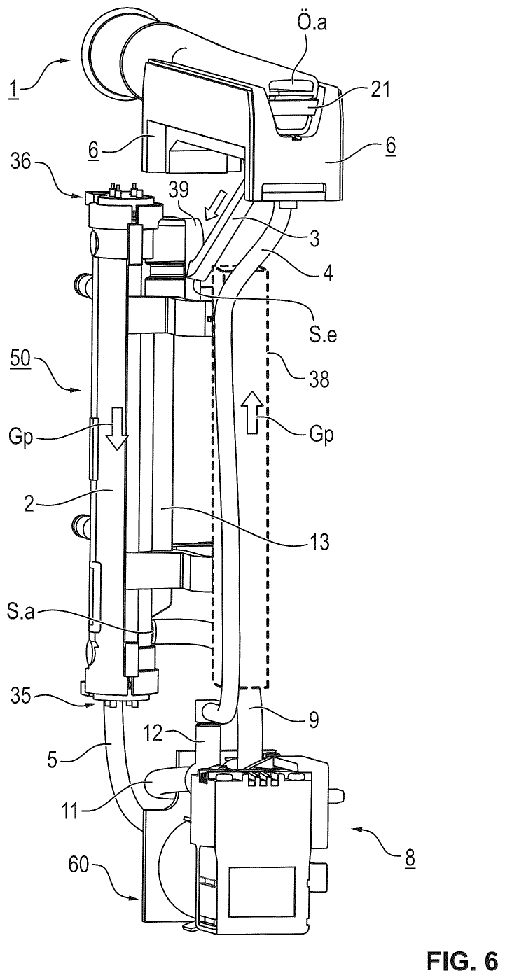

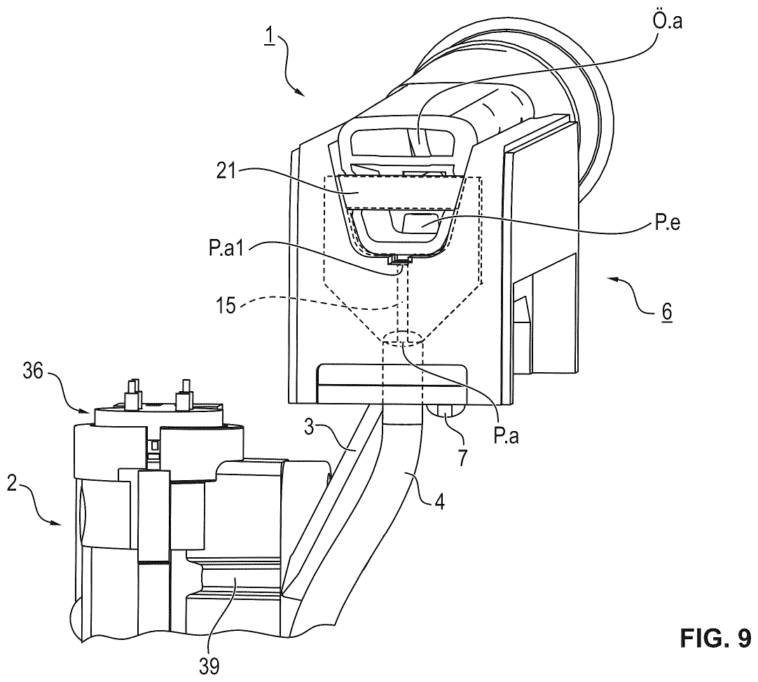

In the embodiment example, the inlet opening Ö.a is circular, while the outlet opening Ö.a has the shape of a trapezoid with rounded corners, see FIG. 6 and FIG. 9.

The mouthpiece 1 is detachably connected to an approximately cuboid base body. The base body comprises a two-part housing, of which the cover 6 is shown in perspective and a base part 16 is only shown schematically. The mouthpiece 1 is snapped into the cover 6 and can be removed again from the cover 6. A human being holds the base part 16 in one hand and holds the inlet opening Ö.e in front of the test subject's mouth so that the test subject can input the breath sample A into the mouthpiece 1. At least part of the breath sample A exits through the outlet opening Ö.a and is released back into the environment. It is possible that the rest of the breath sample A flows past the mouthpiece 1 and then escapes into the environment.

The mouthpiece 1 has the shape of a tube. Therefore, the base part 16 and thus also the mouthpiece 1 can be positioned and oriented in such a way that the breath sample A emerging from the mouthpiece 1 flows in a desired direction. As a result, the risk of the exhaled breath sample A flowing into the face of the test subject or another human being in the vicinity is relatively low.

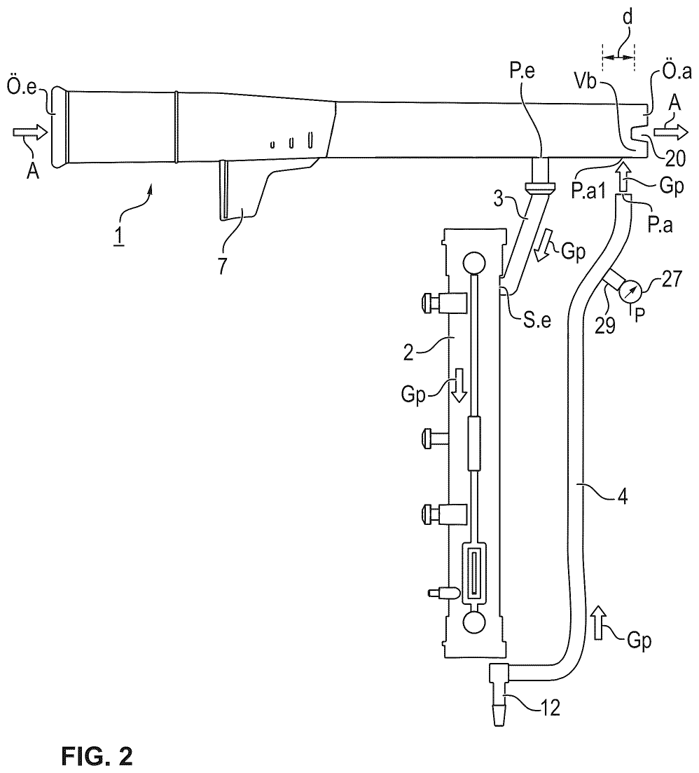

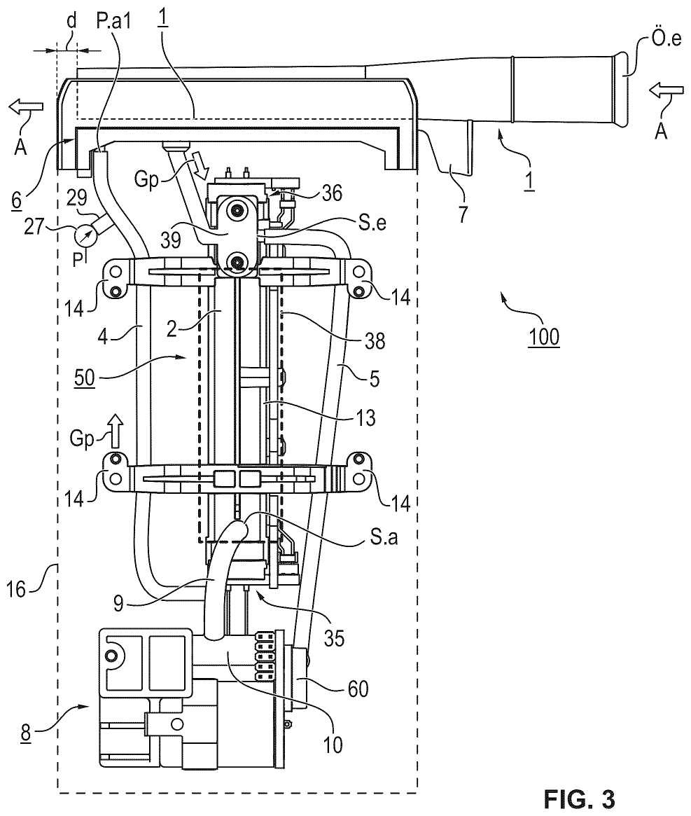

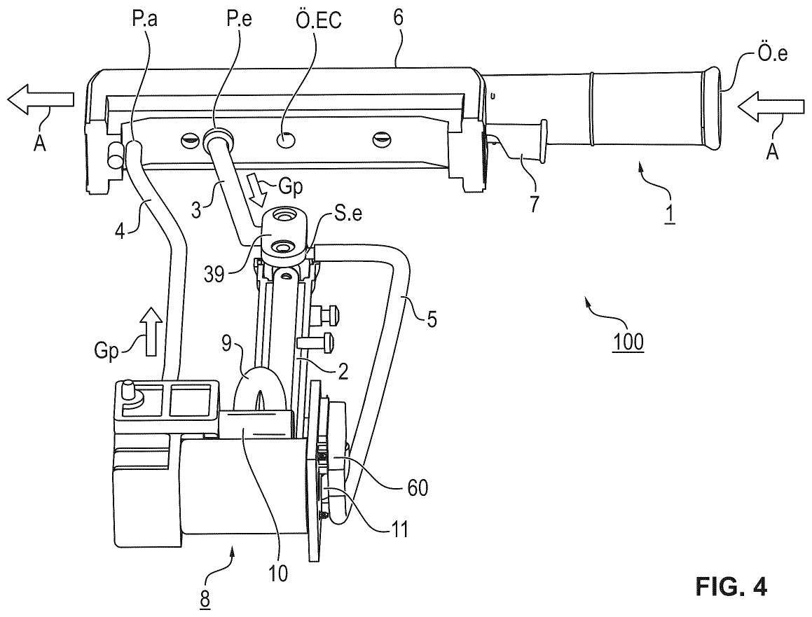

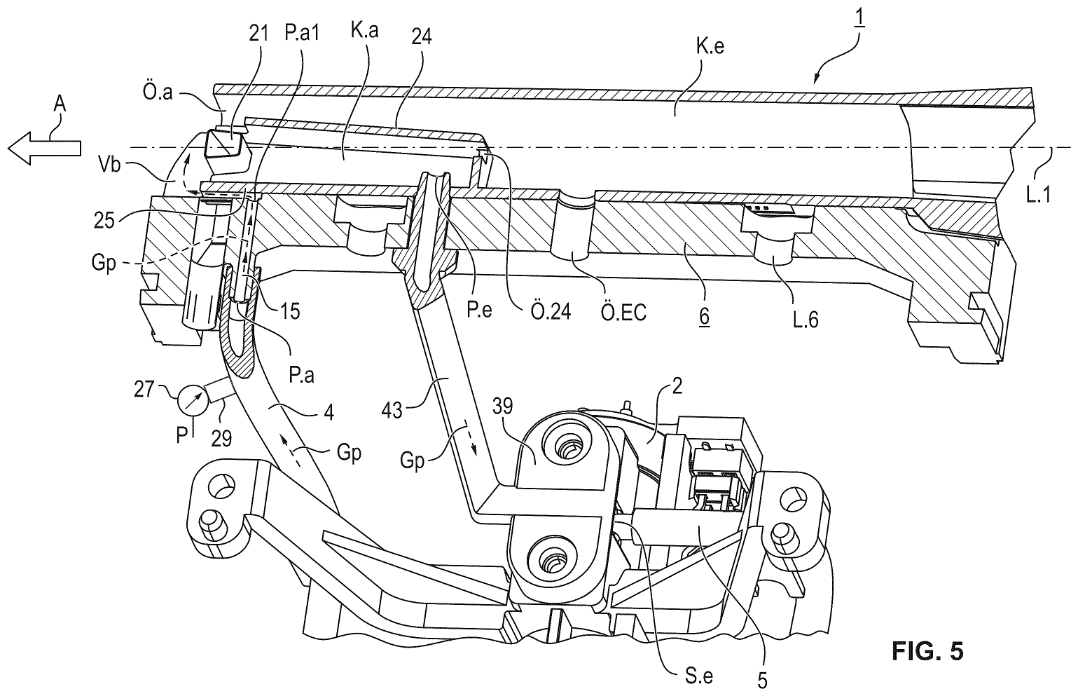

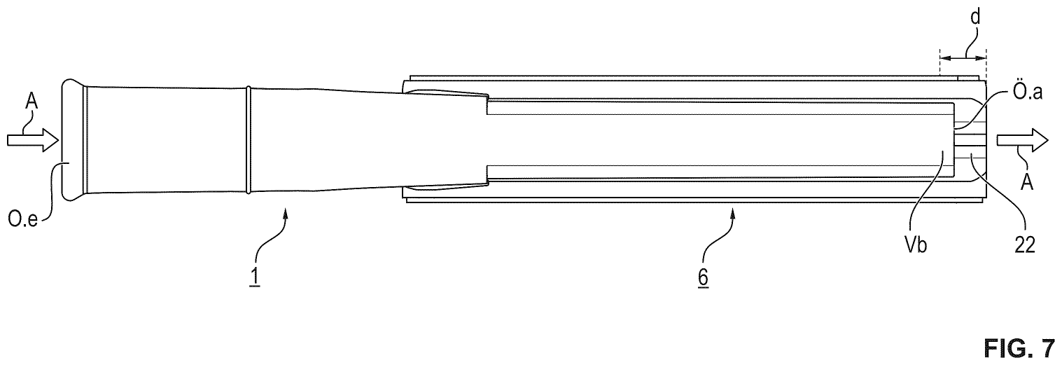

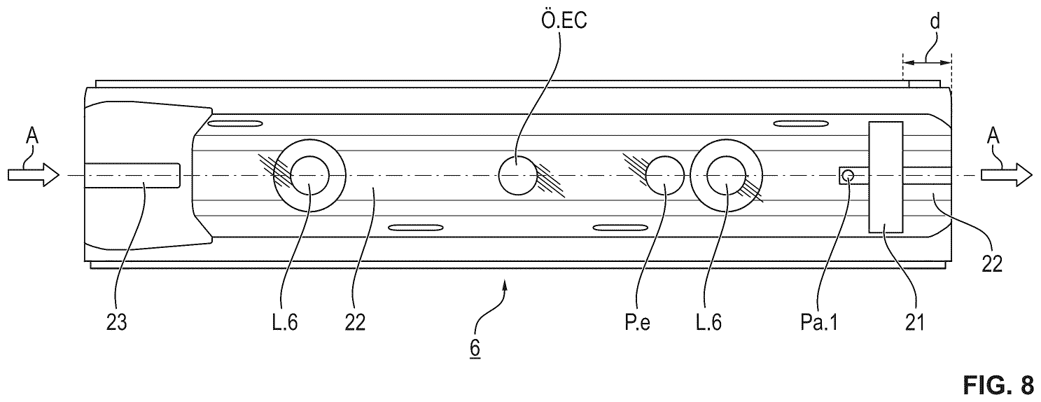

FIG. 1 shows the alcohol measuring device 100 in a side view from the right, i.e. the breath sample A flows through the mouthpiece 1 from left to right. FIG. 2 shows the alcohol measuring device 100 of FIG. 1, with the base body 6, 16 omitted. FIG. 3 shows the alcohol measuring device 100 in a side view from the left, i.e. the breath sample A flows from right to left through the mouthpiece 1. FIG. 4 shows the alcohol measuring device 100 in a view from the left and from diagonally below, i.e. the breath sample A flows from right to left through the mouthpiece 1. FIG. 5 shows a sectional view, the viewing direction being from the left. FIG. 6 shows the alcohol measuring device 100 in an oblique view from the right, i.e. the breath sample A flows obliquely towards the viewer. FIG. 7 shows the mouthpiece 1 and the cover 6 from above, with the other components omitted. FIG. 8 shows the cover 6 from above, with the mouthpiece 1 also omitted. In FIG. 7 and FIG. 8 it can be seen that the cover 6 protrudes downstream of the mouthpiece 1 by the distance d above the mouthpiece 1, namely at the outlet opening Ö.a. FIG. 9 shows a cross-section through the alcohol measuring device 100 in a viewing direction obliquely from behind, namely at the level of a tube 15 explained further below.

A separating element 24 divides the mouthpiece 1 into an inlet chamber K.e and an outlet chamber K.a, see FIG. 5. The inlet chamber K.e is adjacent to the inlet opening Ö.e and the outlet chamber K.a is adjacent to the outlet opening Ö.a. The separating element 24 separates the two chambers K.e and K.a from one another in a fluid-tight manner, with the exception of an opening Ö.24. When the subject delivers a breath sample A, dynamic pressure occurs in the inlet chamber K.e. A pressure sensor, which is not shown, measures an indicator of this dynamic pressure. The progression of this dynamic pressure over time indicates whether and, if so, when and for how long the test subject inputs a breath sample A into the mouthpiece 1. Thanks to the separating element 24, a pressure occurs in the outlet-side chamber K.a that deviates only slightly from the ambient pressure.

In the embodiment shown in FIG. 5, the inlet-side chamber K.e extends from the inlet opening Ö.e to the outlet opening Ö.a. In one embodiment, the outlet-side chamber K.a is separated from the outlet opening Ö.a by a wall. Viewed in the flow direction in which the breath sample A flows through the input unit 1, the chamber K.a on the outlet side is located next to the segment of the chamber K.e on the inlet side that is adjacent to the outlet opening Ö.a.

A projection 7 on the mouthpiece 1 engages in a corresponding slot 23 in the cover 6, see FIG. 8. Two opposing slots 20 are arranged in the mouthpiece 1 at the outlet opening Ö.a. A web 21 of the cover 6 engages in these slots 20 when the mouthpiece 1 is inserted, see FIG. 6 and FIG. 9.

Part of the breath sample A passes from the outlet-side chamber K.a of the mouthpiece 1 into a measuring cuvette 2, which acts as the measuring chamber. This part is referred to below as the “gas sample Gp”. The measuring cuvette 2 has approximately the shape of a cylinder and extends along a longitudinal axis L.2, which in the embodiment example is perpendicular or oblique to the longitudinal axis L.1 of the mouthpiece 1. When a human being holds the base body 6, 16 in his/her hand, the longitudinal axis L.2 is arranged approximately vertically. As can best be seen in FIG. 6, the measuring cuvette 2 is offset slightly to the right relative to the mouthpiece 1 and the cover 6. Preferably, the measuring cuvette 2 has an inner wall that reflects electromagnetic radiation. The wall is made of a material that ideally does not react chemically with the gas sample, for example gold or an alloy containing gold.

The measuring cell 2 is surrounded by a casing (sheath) 13. The casing 13 is attached to an inner wall of the base body 6, 16 with the aid of fastening elements 14. The casing 13 is omitted in several figures.

At least one gas sensor measures the breath alcohol content in the gas sample Gp, which is located in the measuring cuvette 2. Various principles of how the gas sensor can work can be applied, such as an electrochemical sensor, an infrared sensor, an ionizing sensor or a photoacoustic sensor.

In the embodiment example, the alcohol measuring device 100 comprises a photoelectric sensor 50. The sensor 50 comprises the measuring cuvette 2, a radiation source 35, a photodetector 36 and optionally a wavelength filter (not shown). Preferably, the radiation source 35 and the photodetector 36 are attached to two opposite end walls of the measuring cuvette 2 in order to achieve a large optical path. In the embodiment example, the radiation source 35 is mounted at the bottom and the photodetector 36 at the top.

The radiation source 35 emits electromagnetic radiation, in particular infrared radiation, into the measuring cuvette 2. The electromagnetic radiation penetrates the measuring cuvette 2 at least once and strikes the photodetector 36. The photodetector 36 generates an analog signal or digital signal, which depends on the intensity of the incident radiation. Breath alcohol in the measuring cuvette 2 absorbs part of the electromagnetic radiation in a predetermined wavelength range. Preferably, the wavelength filter only allows radiation in this wavelength range to pass through. This reduces the risk of water droplets in the gas sample Gp falsifying a measurement result.

The greater the concentration of breath alcohol, the more electromagnetic radiation is absorbed in the relevant wavelength range in the measuring cuvette 2. Therefore, the signal of the photodetector 36 is an indicator of the concentration of breath alcohol in the gas sample Gp. An evaluation unit of a signal-processing control unit 60 evaluates the signal of the photodetector 36 and generates a signal which contains information about the content of breath alcohol in the gas sample Gp. Preferably, an output unit of the alcohol measuring device 100, which is not shown, outputs this information in at least one form that can be perceived by a human being.

The gas sample Gp is branched off at an entry point P.e from the breath sample A, which flows through the mouthpiece 1. Preferably, a maximum of 10% by volume of the breath sample A is branched off as a gas sample, particularly preferably a maximum of 2% by volume. The gas sample Gp flows through a feed line 3 and a connecting piece 39 into the measuring cuvette 2, through the measuring cuvette 2 and through a discharge line 4 back out of the measuring cuvette 2 and in a first alternative back into the mouthpiece 1. In the embodiment example, the feed line 3 is a rigid tube and is heated, for example by heating elements in the wall of the tube 3 and/or in the connecting piece 39. The discharge line 4 is a flexible hose.

FIG. 1 to FIG. 6 use arrows to illustrate the path of the gas sample Gp. The gas sample Gp inputs the feed line 3 at the entry point P.e and flows through the feed line 3, through the measuring cuvette 2 and the discharge line 4 to an exit point P.a, see FIG. 2 and FIG. 4. The gas sample then flows through a tube 15 in the cover 6 to an exit point P.a1. In a first alternative, the gas sample Gp re-enters the mouthpiece 1 at the exit point P.a1, see FIG. 2 and FIG. 9. FIG. 9 shows a cross-section through the gas measuring device 100 at the height of the exit point P.a1, whereby the longitudinal axis L.1 of the mouthpiece 1 is almost perpendicular to the plane of the drawing.

In a second alternative, the gas sample Gp does not flow back into the mouthpiece 1. Rather, the gas sample Gp flows from the exit point P.a1 in a slot 25 between the mouthpiece 1 and the cover 6 in the direction of the outlet opening Ö.a. The gas sample Gp thus flows past the mouthpiece 1 into an environment of the alcohol measuring device 100.

In both alternatives, the exit point P.a1 is located at a distance downstream of the entry point P.e.

In the embodiment example, the alcohol measuring device 100 comprises, in addition to the photoelectric sensor 50 just described with the measuring cuvette 2, the radiation source 35 and the photodetector 36, an electrochemical sensor 38, which is only shown schematically. An additional gas sample emerges from the mouthpiece 1 through an opening Ö.EC in the cover 6 and reaches a measuring chamber of the electrochemical sensor 38, which is not shown. A chemical reaction takes place in this measuring chamber, which influences an electrical detection variable. Preferably, the chemical reaction is the one that also takes place in a fuel cell. This electrical detection variable is measured and is an indicator of the concentration of breath alcohol in the other gas sample.

The feature that the alcohol measuring device 100 comprises both a photoelectric sensor 50 and an electrochemical sensor 38 has the following advantages in particular:
The two sensors provide redundancy. If one gas sensor 50 or 38 fails, the other gas sensor 38 or 50 can still measure the breath alcohol content in breath sample A.
The measured values of the two sensors 50, 38 can be compared with each other. If the measured values differ greatly, this may indicate that at least one gas sensor 50, 38 is defective.
It is possible that the breath sample A, and therefore the gas sample Gp, contains-besides breath alcohol-another component to which the two gas sensors 50 and 38 react differently. In many cases, this additional component can be reliably distinguished from breath alcohol if two different sensors 50 and 38 are used.
A stream of gas flows through the measuring cuvette 2 so that there is a different gas sample Gp in the measuring cuvette 2 at any time. The photoelectric sensor 50 measures the breath alcohol content continuously, or more precisely: with a fixed sampling frequency. The signal of the photoelectric sensor 50 therefore provides a time course of the breath alcohol in the gas sample Gp. This makes it possible in many cases to distinguish the breath alcohol content in different parts of the breath sample A from one another. Breath sample A comprises a part that comes from the mouth of the test subject, then a part coming from the upper respiratory tract and finally a part coming from the lungs. It is often desired to know whether a measured value for the alcohol content refers to a breath sample from the mouth, the upper respiratory tract or the lungs. Due to its measuring principle, the electrochemical sensor 38 is only able to measure the breath alcohol content at a lower sampling frequency than the photoelectric sensor 50.

The following description again refers to the gas sample Gp, which is examined in the photoelectric sensor 50. The same applies to the other gas sample which is examined by the optional electrochemical sensor 38. The gas sample Gp mixes in a mixing region Vb with that part of the breath sample A which has flowed from the inlet opening Ö.e through the mouthpiece 1 to the outlet opening Ö.a without being branched off. In the first alternative, this mixing region Vb in the mouthpiece 1 extends from the exit point P.a1 to the outlet opening Ö.a, see FIG. 2 and FIG. 9. In the second alternative, the mixing region Vb is arranged downstream of the mouthpiece 1 and above the channel 22, see FIG. 5 and FIG. 7.

The second alternative has the following advantage in particular: The mixing region Vb, in which the gas sample Gp mixes with the rest of the breath sample A, is located downstream of the mouthpiece 1 in the second alternative and therefore outside the mouthpiece 1. Therefore, the risk of turbulence and/or back pressure occurring in the outlet-side chamber K.a of the mouthpiece 1 is reduced.

Several openings are recessed into the cover 6, including
an opening for the entry point P.e, where the feed line 3 to the photoelectric sensor 50 begins,
an opening Ö.EC, in which an unguided feed line to the electrochemical sensor 38 begins, and
in the first alternative, an opening for the exit point P.a1, into which the tube 15 opens, see FIG. 8 and FIG. 9.

In addition, several holes L.6 for a screw connection with the base part 16 are embedded in the cover 6, see FIG. 5 and FIG. 8.

A channel 22 is recessed in the cover 6, see FIG. 7 and FIG. 8. This channel 22 extends along the entire longitudinal axis L.6 of the cover 6. If the mouthpiece 1 is placed on the cover 6, the mouthpiece 1 engages in the channel 22 and closes several openings in the cover 6, see FIG. 5. The two openings for the entry point P.e and the exit point P.a1 (first alternative only) and the opening Ö.EC are not closed.

It is possible that the excess pressure generated by the subject exhaling in the mouthpiece 1 is sufficient to feed the gas sample into the measuring cuvette 2. In the embodiment example, however, a fluid conveying unit 8 is preferably used to divert the gas sample Gp from the breath sample A flowing through the mouthpiece 1, to deliver it into the measuring cuvette 2 and to expel it from the measuring cuvette 2 again. The fluid conveying unit 8 draws gas out of the measuring cuvette 2 through a line 9. This creates a vacuum and the gas sample Gp flows through the feed line 3 into the measuring cuvette 2. In many cases, the gas sample Gp fills the measuring cuvette 2 relatively evenly, which increases the reliability of the measurement. The aspirated gas sample Gp flows from the measuring cuvette 2 through a hose 9 and a rigid line 10 into a chamber of the fluid conveying unit 8. The gas sample Gp then flows from the chamber of the fluid conveying unit 8 through a tube 12 into the discharge line 4.

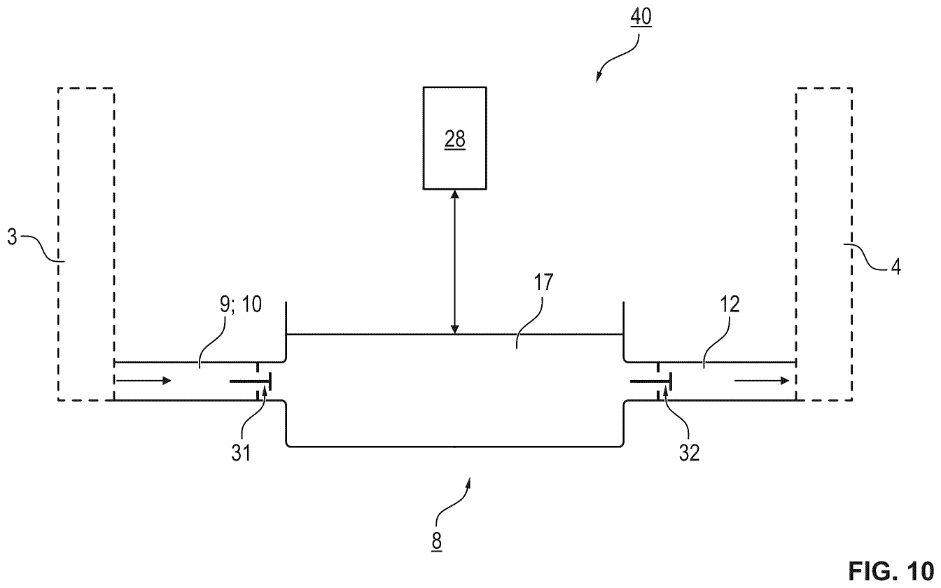

Various configurations of the fluid conveying unit 8 are possible. In the embodiment example, the fluid conveying unit 8 comprises a diaphragm pump 40, which is shown schematically in FIG. 10. The diaphragm pump 40 comprises a chamber 17 with a variable volume and an actuator 28, shown schematically, which is able to change the volume of the chamber 17. In this schematic representation, the feed 9, 10 opens into the chamber 17 from the left, and the discharge 12 leads out of the chamber 17 on the right. An intake non-return valve 31 releases a flow of gas into the chamber 17 and blocks a flow in the opposite direction. Similarly, an output non-return valve 32 releases a flow of gas out of the chamber 17 and blocks a flow in the opposite direction.

In the embodiment, the volume flow of the gas sample Gp is measured through the measuring cuvette 2. By integrating over time, the control unit 60 uses the measured volume flow to approximately derive the volume that has flowed into the measuring cuvette 2 since a certain event, for example since the mouthpiece 1 was fitted. A measurement result of the alcohol measuring device 100 is only valid if a gas sample Gp with a sufficiently large volume has entered the measuring cuvette 2.

The “volume flow”, also called volume flow rate, is understood to be the volume per unit time of the fluid that flows through a fluid guide unit, for example through the measuring cuvette 2. To measure the volume flow, a time-varying pressure difference is measured at a specific sampling frequency, namely a difference between the pressure at the point S.e, at which the fluid exits the feed line 3 and enters the measuring cuvette 2, and the pressure at the point S.a, at which the fluid exits the measuring cuvette 2 and enters the line 9, see FIG. 3. This pressure difference is an indicator of the volume flow. The following describes an implementation process for measuring this pressure difference.

A measuring line 5 is in a fluid connection with the outlet opening of the feed line 3. Therefore, the same pressure prevails in the measuring line 5 as at the point S.e at which the fluid enters the measuring cell 2. The same pressure prevails in the line 9 as at the point S.a where the fluid exits the measuring cuvette 2. A sensor for the pressure difference is in a fluid connection with lines 9 and 5. Measured values relating to the pressure difference are transmitted to the control unit 60, and the control unit 60 derives the volume flow from these measured values and generates a signal comprising the measured volume flow. A line 11 pneumatically connects the measuring line 5 to the line 10.

After the breath sample A of a test subject has been examined, it is necessary to rinse out the measuring cuvette 2. In the embodiment example, the fluid conveying unit 8 is also used to rinse out the measuring cuvette 2. During rinsing, ambient air flows through the measuring cuvette 2 along the same path as a gas sample Gp to be analyzed. The measuring cuvette 2 can be rinsed with the mouthpiece 1 connected or without the mouthpiece 1. Preferably, the volume flow is also measured during rinsing to ensure that a sufficient amount of ambient air flows through the measuring cuvette 2 during rinsing.

It is also possible that the fluid conveying unit 8 is used exclusively to rinse out the measuring cuvette 2. As already explained, in one embodiment, the pressure with which a test subject delivers the breath sample A is sufficient for the gas sample Gp to be examined to enter the measuring cuvette 2. The embodiment of using a fluid conveying unit 8 exclusively for rinsing the measuring cuvette 2 makes it possible to use a fluid conveying unit that takes up less installation space and consumes less electrical energy compared to a fluid conveying unit that draws in a breath sample A.

The breath alcohol content can only be measured correctly if the mouthpiece 1 is correctly placed on the cover 6 and snaps into place. The mouthpiece 1 then engages in the channel 22. Preferably, the fluid conveying unit 8 should be switched on both when the gas sample Gp is branched off from the breath sample A and when the measuring cuvette 2 is rinsed. The embodiment described below makes it possible to automatically check whether these conditions are currently fulfilled or not.

A lower pressure limit value x and a higher pressure limit value y are specified. A pressure sensor 27 is capable of measuring an indicator of the pressure in the discharge line 4. This pressure sensor 27 can belong to the sensor for the pressure difference described above, whereby the pressure difference is measured in order to measure the volume flow into and out of the measuring cuvette 2. The pressure sensor 27 is pneumatically connected to the discharge line 4 via a connecting line 29.

The control unit 60 compares the measured pressure with the two pressure limit values x and y. If the measured pressure is less than the smaller pressure limit value x, the fluid conveying unit 8 is switched off. If the measured pressure is between the two pressure limit values x and y, the fluid conveying unit 8 is switched on and no mouthpiece 1 is fitted on the cover 6. Or the mouthpiece 1 is not fitted correctly, for example not engaged, so that there is too great a gap between the mouthpiece 1 and the cover 6. If the measured pressure is greater than the greater pressure limit value y, the fluid conveying unit 8 is switched on and a mouthpiece 1 is fitted. The reason for this distinction: If a mouthpiece 1 is fitted, a greater pressure occurs in the discharge line 4 than if no mouthpiece 1 is fitted. The pressure limit values x and y are preferably determined empirically in advance.

Preferably, the alcohol measuring device 100 outputs a message in a form that can be perceived by a human being if the alcohol measuring device 100 is to take and examine a breath sample A, but a mouthpiece 1 is not fitted at all or is not fitted correctly.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Tubular mouthpiece, which can be detachably fitted onto the |
| | cover 6, comprises the projection 7 and the recess 21, |
| | extends along the longitudinal axis L.1, detachably |
| | connected to the cover 6, functions as the input unit |
| 2 | Measuring cuvette, belongs to the photoelectric sensor 50, |
| | extends along the longitudinal axis L.2, functions as the |
| | measuring chamber |
| 3 | Feed line, belongs to the feed fluid guide unit |
| 4 | Discharge line, belongs to the discharge fluid guide unit |
| 5 | Measuring line |
| 6 | Cover of the alcohol measuring device 100, carries the |
| | mouthpiece 1, forms the base body of the alcohol measuring |
| | device 100 together with the base part 16, has the holes L.6 |
| 7 | Projection on the mouthpiece 1, engages in the slot 23 |
| 8 | Fluid conveying unit, draws the gas sample Gp from the |
| | mouthpiece 1, in one embodiment configured as a |
| | diaphragm pump 40 |
| 9 | Line from the line 10 to the measuring cell 2 |
| 10 | Line from chamber 17 to line 9 |
| 11 | Line, connects the measuring line 5 with the line 10 |
| 12 | Tube, leads from chamber 17 to discharge line 4 |
| 13 | Casing of the measuring cuvette 2 |
| 14 | Fastening elements for the casing 13 |
| 15 | Tube in cover 6 |
| 16 | The base part, together with the cover 6, forms the base |
| | body of the alcohol measuring device 100 |
| 17 | Chamber of the diaphragm pump 40 |
| 20 | Slots in the mouthpiece 1 at the outlet opening Ö.a |
| 21 | Web of the cover 6, engages in the slots 20 |
| 22 | Channel in cover 6 |
| 23 | Slot in the cover 7, into which the projection 7 engages |
| 24 | Separating element, which divides the mouthpiece 1 into an |
| | inlet-side chamber K.e and an outlet-side chamber K.a, has |
| | the opening Ö.24 |
| 27 | Pressure sensor, measures the pressure in the discharge line 4 |
| 28 | Diaphragm pump actuator 40 |
| 29 | Connection line between the pressure sensor 27 and the |
| | discharge line 4 |
| 31 | Intake non-return valve of the diaphragm pump 40 |
| 32 | Output non-return valve of the diaphragm pump 40 |
| 35 | Radiation source of the photoelectric sensor 50 |
| 36 | Photodetector of the photoelectric sensor 50 |
| 38 | Electrochemical sensor |
| 39 | Connecting piece between the feed line 3 and the |
| | measuring cell 2 |
| 40 | Diaphragm pump, comprises the chamber 17, the |
| | actuator 28, the intake non-return valve 31 and |
| | the output non-return valve 32 |
| 50 | Photoelectric sensor, comprises the measuring cell 2, the |
| | radiation source 35 and the photodetector 36 |
| 60 | Control unit of the alcohol measuring device 100 |
| 100 | Alcohol measuring device, comprises the mouthpiece 1, |
| | the base part 16, the cover 6, the sensors 50 and 38, |
| | the fluid conveying unit 8, the lines 3, 4, 5, 9, 10, 11 |
| | and the control unit 60 |
| A | Breath sample, inputs the mouthpiece through the inlet |
| | opening Ö.e and leaves through the outlet opening Ö.a |
| | of the mouthpiece 1 |
| d | Distance over which the cover 6 protrudes beyond the |
| | mouthpiece 1 |
| Gp | Gas sample, flows through the feed line 3 into the |
| | measuring cuvette 2, through the measuring cuvette 2 |
| | and through the discharge line 4 |
| K.a | Outlet-side chamber in mouthpiece 1 |
| K.e | Inlet-side chamber in mouthpiece 1 |
| L.1 | Longitudinal axis of mouthpiece 1 |
| L.2 | Longitudinal axis of the measuring cell 2 |
| L.6 | Holes in the cover 6 for a screw connection with the |
| | base part 16 |
| Ö.e | Inlet opening of the mouthpiece 1 |
| Ö.a | Outlet opening of the mouthpiece 1 |
| Ö.24 | Opening in separating element 24 |
| Ö.EC | Opening in the cover 6, in which a feed line to the |
| | electrochemical sensor 38 begins |
| P.a | Exit point from the discharge line 4 |
| P.e | Entry point into the feed line 3 |
| P.a1 | Exit point from the tube 15 |
| S.a | Position at the end of the line 9 |
| S.e | Point at which the measuring cell 2 joins the line 9 |
| Vb | Mixing region in which the branched-off gas sample Gp |
| | mixes with the rest of the breath sample A |
| x | Lower pressure limit |
| y | Higher pressure limit |

What is claimed is:

1. A gas measuring device for measuring a concentration of at least one component in a gas mixture, the gas measuring device comprising:
a base body;
a tubular input unit with an inlet opening and an outlet opening, the tubular input unit being configured to be connected to the base body;
a measuring chamber arranged in an interior of the base body;
a feed fluid guide unit connecting an inlet point of the base body to the measuring chamber;
a discharge fluid guide unit connecting the measuring chamber to an exit point of the base body, wherein the discharge fluid guide unit is spaced apart from the feed fluid guide unit; and
a gas sensor arrangement comprising at least one gas sensor,
wherein the gas measuring device is configured such that a gas mixture flows through the inlet opening into the input unit and through the input unit, a gas sample is branched-off at the entry point from the gas mixture flowing through the input unit, and a remaining part of the gas mixture flows past the entry point,
wherein the gas measuring device is configured such that the branched-off gas sample flows from the entry point through the feed guide unit into the measuring chamber and flows out of the measuring chamber through the discharge fluid guide unit to the exit point,
wherein the gas measuring device is configured such that the branched-off gas sample flows back into the input unit at the exit point and the branched-off gas sample with the remaining part of the gas mixture as an entire gas mixture leaves the input unit through the outlet opening or wherein the gas measuring device is configured such that the branched-off gas sample flows out of the base body at the exit point and past the input unit in a direction away from the inlet opening, and
wherein the gas sensor arrangement is configured to measure the concentration of the component in the gas sample in the measuring chamber.

2. A gas measuring device according to claim 1,
wherein the gas measuring device is configured such that with the input unit connected to the base body, a channel is formed between the base body and the input unit;
wherein the channel connects the exit point with an environment of the gas measuring device, and
wherein the gas measuring device is configured such that the branched-off gas sample flows from the exit point through the channel.

3. A gas measuring device according to claim 1, wherein the base body, with respect to a flow direction of the gas mixture through the input unit, protrudes downstream of the input unit over the input unit such that a region is formed downstream of the outlet opening, which region is limited on one side by the input unit and on another side by the base body.

4. A gas measuring device according to claim 1, further comprising a separating element dividing the input unit into an inlet side chamber and an outlet side chamber,
wherein the inlet opening is adjacent to the inlet-side chamber and the outlet opening is adjacent to the outlet-side chamber,
wherein at least one passage opening in the separating element connects the inlet-side chamber and the outlet-side chamber, and
wherein the entry point is adjacent to the outlet-side chamber and the exit point is adjacent to the outlet-side chamber.

5. A gas measuring device according to claim 4,
wherein the inlet-side chamber connects the inlet opening with the outlet opening, and
wherein a segment of the inlet-side chamber is located next to the outlet-side chamber, with respect to a flow direction of the gas mixture through the input unit.

6. A gas measuring device according to claim 1, further comprising a pressure sensor,
wherein the input unit is configured to be detachably connected to the base body;
wherein the pressure sensor is configured to measure a pressure in the feed fluid guide unit or a pressure in the discharge fluid guide unit, and
wherein the gas measuring device is configured to determine, depending on a measured pressure, whether the input unit is connected to the base body or not.

7. A gas measuring device according to claim 1, further comprising:
a pressure sensor; and
a fluid conveying unit,
wherein the fluid conveying unit is configured to be switched on and off,
wherein the switched-on fluid conveying unit is configured to convey the gas sample out of the measuring chamber and through the discharge fluid guide unit,
wherein the pressure sensor is configured to measure a pressure in the discharge fluid guide unit, and
wherein the gas measuring device is configured to determine, depending on the measured pressure, whether the fluid conveying unit is switched on or off, and/or to measure a volume flow achieved by the fluid conveying unit.

8. A gas measuring device according to claim 1, further comprising a fluid conveying unit,
wherein the fluid conveying unit comprises:
a chamber with a variable volume,
an actuator configured to change the volume of the chamber;
an intake non-return valve; and
an output non-return valve,
wherein the chamber of the fluid conveying unit is in an intake fluid connection with the measuring chamber and in a discharge fluid connection with the discharge fluid guide unit,
wherein the intake non-return valve is configured to allow a flow of fluid through the intake fluid connection into the chamber and to prevent a flow of fluid from the chamber into the intake fluid connection, and
wherein the output non-return valve is configured to allow a flow of fluid from the chamber into the discharge fluid connection and to prevent a flow of fluid from the discharge fluid connection into the chamber.

9. A gas measuring device according to claim 1, wherein the gas sensor arrangement comprises:
a radiation source configured to emit electromagnetic radiation; and
a photodetector configured to generate a signal depending on an intensity of impinging radiation,
wherein the gas measuring device is configured such that emitted radiation penetrates at least once the measuring chamber and impinges on the photodetector and the generated signal contains information about the concentration of the component of the gas sample.

10. A gas measuring device according to claim 9,
wherein the gas sensor arrangement further comprises an additional gas sensor which has an electrical detection variable,
wherein the gas measuring device is configured such that the branched-off gas sample reaches both the gas sensor with the radiation source and the additional gas sensor or wherein the gas measuring device is configured such that an additional gas sample is branched off from the gas mixture and reaches the additional gas sensor,
wherein the additional gas sensor is configured such that a chemical reaction takes place in the additional gas sensor and the chemical reaction influences the electrical detection variable,
wherein the chemical reaction and the electrical detection variable depend on the concentration of the component in the gas sample or in the additional gas sample, and
wherein the additional gas sensor is configured to measure the electrical detection variable and to generate a signal depending on the measured electrical detection variable such that the generated signal comprises information about the concentration of the component in the gas sample or in the additional gas sample.

11. A process for measuring a concentration of a component in a gas mixture, the process comprising the steps of:
providing a gas measuring device, wherein the gas measuring device comprises a tubular input unit, a base body, a feed fluid guide unit, a measuring chamber, a discharge fluid guide unit and a sensor arrangement comprising at least one gas sensor, wherein the input unit has an inlet opening and an outlet opening and is at least temporarily connected to the base body, wherein the measuring chamber is arranged in an interior of the base body, and wherein the two fluid guide units are arranged at a distance from one another in the base body;
allowing or causing the gas mixture to flow through the inlet opening into the input unit and through the input unit;
branching off at an entry point of the base body a gas sample from the gas mixture flowing through the input unit and allowing a remaining part of the gas mixture to flow past the entry point;
allowing the branched-off gas sample to flow back at an exit point of the base body into the input unit and allowing an entire gas mixture to leave the input unit through the outlet opening or allowing the branched-off gas sample to flow at the exit point out of the base body and to pass the input unit in a direction away from the inlet opening;
allowing the branched-off gas sample to flow from the entry point through the feed guide unit into the measuring chamber;
with the gas sensor arrangement, measuring the concentration of the component in the gas sample in the measuring chamber; and
allowing the gas sample to flow from the measuring chamber through the discharge fluid guide unit to the exit point.

12. A process according to claim 11,
wherein the gas measuring device is configured such that with the input unit connected to the base body, a channel is formed between the base body and the input unit,
wherein the channel connects the exit point with an environment of the gas measuring device, and
wherein the process comprises the further step that the gas sample flows from the exit point through the channel.

13. A process according to claim 11, wherein the base body, with respect to a flow direction of the gas mixture through the input unit, protrudes downstream of the input unit over the input unit such that a region is formed downstream of the outlet opening, which region is limited on one side by the input unit and on another side by the base body.

14. A process according to claim 11,
wherein the gas measuring device further comprises a separating element dividing the input unit into an inlet side chamber and an outlet side chamber,
wherein the inlet opening is adjacent to the inlet-side chamber and the outlet opening is adjacent to the outlet-side chamber,
wherein at least one passage opening in the separating element connects the inlet-side chamber and the outlet-side chamber, and
wherein the entry point is adjacent to the outlet-side chamber and the exit point is adjacent to the outlet-side chamber.

15. A process according to claim 14,
wherein the inlet-side chamber connects the inlet opening with the outlet opening, and
wherein a segment of the inlet-side chamber is located next to the outlet-side chamber, with respect to a flow direction of the gas mixture.

16. A process according to claim 11, wherein the gas measuring device further comprises a pressure sensor and the process further comprises:
configuring the input unit to be detachably connected to the base body;
with the pressure sensor measuring a pressure in the feed fluid guide unit or a pressure in the discharge fluid guide unit, and
determining, depending on a measured pressure, whether the input unit is connected to the base body or not.

17. A process according to claim 11, wherein the gas measuring device further comprises: a pressure sensor; and a fluid conveying unit, and the process further comprises:
configuring the fluid conveying unit to be switched on and off, wherein the switched-on fluid conveying unit is configured to convey the gas sample out of the measuring chamber and through the discharge fluid guide unit;
with the pressure sensor, measuring a pressure in the discharge fluid guide unit; and
determining, depending on the measured pressure, whether the fluid conveying unit is switched on or off, and/or determining a volume flow achieved by the fluid conveying unit.

18. A process according to claim 11,
wherein the gas measuring device further comprises a fluid conveying unit,
wherein the fluid conveying unit comprises a chamber with a variable volume, an actuator configured to change the volume of the chamber, an intake non-return valve, and an output non-return valve,
wherein the chamber is in an intake fluid connection with the measuring chamber and in a discharge fluid connection with the discharge fluid guide unit,
wherein the intake non-return valve is configured to allow a flow of fluid through the intake fluid connection into the chamber and to prevent a flow of fluid from the chamber into the intake fluid connection, and
wherein the output non-return valve is configured to allow a flow of fluid from the chamber into the discharge fluid connection and to prevent a flow of fluid from the discharge fluid connection into the chamber.

19. A process according to claim 11, wherein the gas sensor arrangement comprises a radiation source, and a photodetector, and
wherein the process further comprises:
with the radiation source, emitting electromagnetic radiation,
configuring or enabling the gas measuring device such that emitted radiation penetrates the measuring chamber and impinges on the photodetector, and
with the photo detector, generating a signal depending on an intensity of impinging radiation
wherein the generated signal contains information about the concentration of the component of the gas sample.

20. A process according to claim 19, wherein the gas sensor arrangement further comprises an additional gas sensor which has an electrical detection variable, and the process further comprises:
configuring the gas measuring device such that the branched-off gas sample reaches both the gas sensor with the radiation source and the additional gas sensor or such that an additional gas sample is branched off from the gas mixture and reaches the additional gas sensor;
configuring the additional gas sensor such that a chemical reaction takes place in the additional gas sensor and the reaction influences the electrical detection variable, wherein the chemical reaction and the electrical detection variable depend on the concentration of the component in the gas sample or the additional gas sample; and
configuring the additional gas sensor to measure the electrical detection variable and generate a signal depending on the measured electrical detection variable such that the generated signal comprises information about the concentration of the component in the gas sample or the additional gas sample.
